(19) United States
(12) Patent Application Publication
(10) Pub. No.: US 2015/0342645 A1
(43) Pub. Date: Dec. 3, 2015

(54) A Novel Medical Equipment for Anchoring and Closing of Cranial Flap During Neurosurgery of Head

(71) Applicant: Ghansham Das AGARWAL, Kolkata 73 (IN)

(72) Inventor: Ghansham Das Agarwal, Kolkata (IN)

(21) Appl. No.: 14/653,043

(22) PCT Filed: Dec. 18, 2013
(86) PCT No.: PCT/IN2013/000788

§ 371 Date: Jun. 17, 2015

(30) Foreign Application Priority Data
Dec. 19, 2012 (IN) 1430/KOL/2012

(51) Int. Cl. A61B 17/68

(52) U.S. Cl. CPC A61B 17/688; A61B 2017/00831

(57) ABSTRACT

Provided herein is a novel medical equipment for anchoring and closing of cranial flap during neurosurgery of head comprising of at least two discs with a connecting member therebetween, wherein said discs and connecting member are provided integrally with a hole therethrough for discharge of fluid. It is associated with the following advantageous features: cost effective, does not require special instruments for opening, faster to apply, thereby saving cost of operation, facilitates reoperation by just rotating to unlock, and can be kept unlocked unlike prior art.

### FIELD OF INVENTION

This invention relates to a novel medical equipment for anchoring and closing of cranial flap during neurosurgery of head.

### BACKGROUND/PRIOR ART OF INVENTION

Surgical operations inside the head/on the brain are performed after making an opening in the skull i.e. the bony cage around the brain or cranium. Generally adopted method of opening is to create four/three burr holes (holes are made with the help of burrs followed by cutting in between the holes by means of giggly saw or latest osteotomes (powered tools). The hard shell flap/bony flap/cranial flap thus created is either kept attached on one side with the pericranium or kept away from patient by covering in a moist mop or in saline.

After finishing of operation on brain, this flap needs to be reapplied and attached with the skull. There are different methods to attach it.

Conventional method comprises steps of drilling small holes in skull followed by inserting stainless steel wire through these holes. Thereafter, the wire is twisted, cut to size and bent inside.

Another method makes utilization of two absorbable plastic buttons, one under the skull and one above the skull. Both buttons are tied with absorbable thread. This keeps the flap in position.

Still another method comprises application of two metal/titanium discs. One disc which goes under the skull has a serrated central wire, wherein the serrations are directed downwards. The second disc is threaded on the serrated wire, which has a central hole with locking fins and can be moved downwards only. It requires special instruments to pull the wire through second disc, in which excess wire is cut off and second disc is locked on the skull.

Disadvantages:
It has poor stability and grip. There are chances of flap slipping down. The buttons are quite thick and can be felt under skin and sometimes may look ugly if applied on exposed surface of head (forehead etc). Application of these buttons is also time consuming. Further, these are expensive.
It provides good anchorage. At times, reoperation is required due to complication or other reason, wherein reoperation is cumbersome and also requires special and expensive tools to open. It is also time consuming, thereby making surgery very expensive.

Hence, it can be concluded that, the aforesaid prior arts provide only limited stability to cranial flap. Slight push/pressure can push in the flap and damage brain. Therefore, it is required to develop a novel medical equipment for anchoring and closing of cranial flap during neurosurgery of head, which can address the above drawbacks.

### OBJECTS OF INVENTION

An object of this invention is to propose a novel medical equipment for anchoring and closing of cranial flap during neurosurgery of head.

Another object of this invention is to propose a novel medical equipment for anchoring and closing of cranial flap during neurosurgery of head which is very fast to use and apply (less than two minutes).

Still another object of this invention is to propose a novel medical equipment for anchoring and closing of cranial flap during neurosurgery of head which is cost effective.

Still another object of this invention is to propose a novel medical equipment for anchoring and closing of cranial flap during neurosurgery of head which does not need any special instrumentation for application.

Still another object of this invention is to propose a novel medical equipment for anchoring and closing of cranial flap during neurosurgery of head which is robust enough to resist pressure to prevent damage to brain.

Still another object of this invention is to propose a novel medical equipment for anchoring and closing of cranial flap during neurosurgery of head wherein re-opening of craniotomy flap is fast without requiring special instrumentation.

Still another object of this invention is to propose a novel medical equipment for anchoring and closing of cranial flap during neurosurgery of head which is reusable.

Still another object of this invention is to propose a novel medical equipment for anchoring and closing of cranial flap during neurosurgery of head which allows outward movement of cranial flap in case of brain edema.

### SUMMARY OF THE INVENTION

According to this invention, there is provided a novel medical equipment for anchoring and closing of cranial flap during neurosurgery of head comprising of at least two discs with a connecting member therebetween, wherein said discs and connecting member are provided integrally with a hole therethrough for discharge of fluid.

### BRIEF DESCRIPTION OF THE ACCOMPANYING DRAWINGS

Further objects and advantages of this invention will be more apparent from the ensuing description when read in conjunction with the accompanying drawings and wherein:

FIG. 1 shows: right side view of a novel medical equipment according to the present invention.

FIG. 2 shows: left side view of a novel medical equipment according to the present invention.

FIG. 3 shows: bottom view of a novel medical equipment in accordance with the present invention.

FIG. 4 shows: top view of a novel medical equipment in accordance with the present invention.

FIG. 5 shows: cranial flap in locked mode.

### DETAIL DESCRIPTION OF THE INVENTION WITH REFERENCE TO THE ACCOMPANYING DRAWINGS

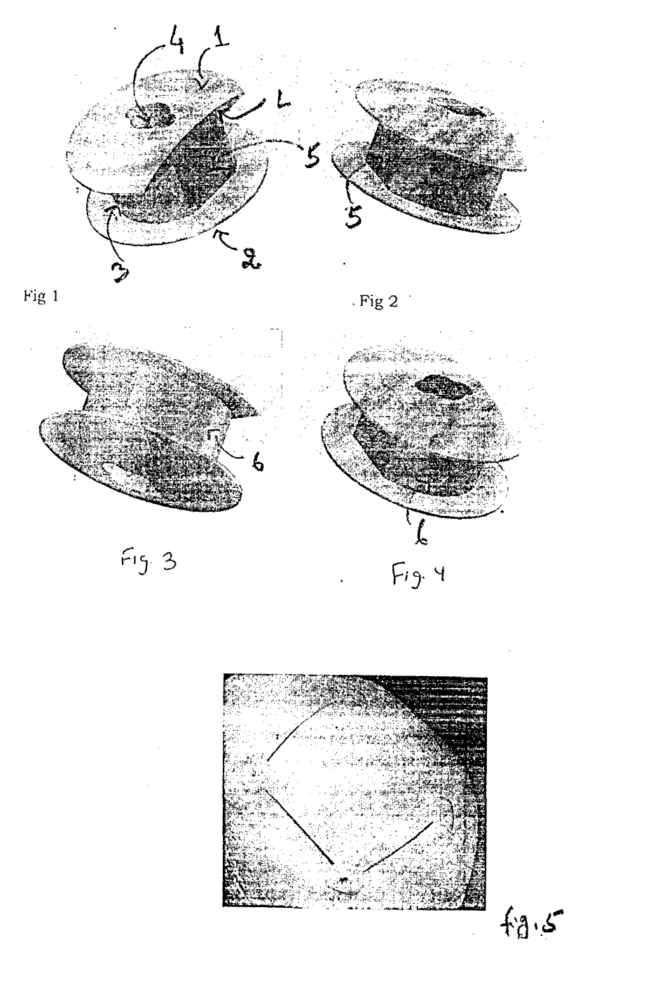

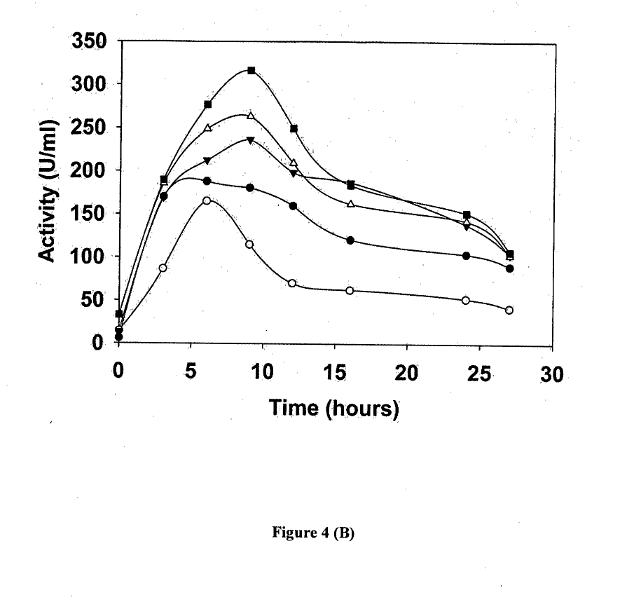

The present invention is directed to a novel medical equipment for anchoring and closing of cranial flap during neurosurgery of head. Now, reference may be made to the accompanying FIGS. 1-4, wherein said equipment comprising of at least two discs (1, 2) connected by means of a connecting member (3) with a hole (4) therethrough.

The top disc (1) comprising of a D-shaped disc and bottom disc (2) is having circular shaped disc.

Further, the top disc is provided with a flat end (L). This helps in rotation of the device to lock and unlock the cranial flap. Again, it allows the cranial flap to be placed in position. Had it been round, it would not have allowed the flap to be placed into position. During placement, the longitudinal side of the disc is inwards. In order to lock it, it is rotated to have round side inwards as shown in FIG. 5.

The bottom disc is provided to hold the flap in position. Further, it does not allow the flap to sink in. The shape is preferably circular because otherwise it can't be locked.

Both the discs are connected by means of the connecting member having two flat surfaces (5) and two cylindrical surfaces (6) provided opposite to each other as shown in FIGS. 1-4.

Two opposite side surfaces of said connecting member are flat for smooth sliding within the burr hole of the cranial flap. Said member obliterates the space of burr hole. Flat side of the top disc and one flat surface of the connecting member are provided in the same plane.

The hole through the equipment allows passage of fluid to outer surface. There is some fluid like water, blood and air under the cranial flap after surgery, which is channeled through said hole. The hole can be of any shape without restricting to the shape indicated in the accompanying figures.

The equipment is preferably made of medical grade plastic, either absorbable or non-absorbable without restricting scope of the invention to the same. It can be manufactured for example by CNC machining of PEEK blanks, injection moulding. Thus, other materials and methods readily apparent to person skilled in the art are understood to be within scope of the invention.

Further, there is provided a method for anchoring and closing of cranial flap comprising steps of: insertion of the equipment into burr hole with two flat sides of the connecting member perpendicular to the direction of insertion followed by rotation of the equipment through an angle of 90° in clockwise direction for locking of the equipment into the burr hole to perform operation.

Closing of the flap comprising steps of resting of the flap on the equipment positioned in four holes followed by rotation of the equipment so that linear side of the top disc faces away from the flap.

The equipment is locked or unlocked by rotation with the help of a screw driver by inserting the screw driver in the hole of said equipment. The cranial flap can't be pushed outward after being locked.

The thickness of the cranial wall is not uniform, therefore the equipment of the present invention is designed according to the size of cranial wall.

The equipment is not rotated (left unlocked) where brain edema is expected because it allows the cranial flap to move outwards not inwards.

The present invention can be left unlocked. However, no other device can be kept unlocked.

When the device is unlocked, the flap can't move in but can move out. This is because the brain is rigid box inside, which does not permit brain to swell. Sometimes after surgery the brain swells like any other organ in body swells after injury/surgery.

In case of present invention, if kept unlocked, it allows the flap to move outward. Thus, it acts as safety valve of pressure cooker. Whenever there is excess pressure inside the skull,, flap can move outward to avoid problems to patient.

Advantageous Features:
Cost effective.
Does not require special instruments for opening
Faster to apply, thereby saving cost of operation.
Facilitates reoperation by just rotating to unlock.
Can be kept unlocked unlike prior art.

It is to be noted that the present invention is susceptible to modifications, adaptations and changes by those skilled in the art. Such variant embodiments employing the concepts and features of this invention are intended to be within the scope of the present invention, which is further set forth under the following claims:

1. A device for anchoring and closing a cranial flap during neurosurgery of head comprising at least two discs with a connecting member therebetween, wherein said discs and connecting member are provided integrally with a hole therethrough for discharge of fluid.

2. The device as claimed in claim 1, wherein at least one of said discs comprises D-shaped disc.

3. The device as claimed in claim 1, wherein at least one of said discs includes a flat end.

4. The device as claimed in claim 1, wherein at least one of said discs is circular shaped.

5. The device as claimed in claim 1, wherein the connecting member comprises two flat surfaces and two cylindrical surfaces provided opposite to each other.

6. The device as claimed in claim 3, wherein the connecting member comprises two flat surfaces and two cylindrical surfaces provided opposite to each other, and wherein the flat end of the at least one disc is parallel to at least one of the flat surfaces of the connecting member.

7. The device as claimed in claim 1, wherein the device has a locked configuration and an unlocked configuration, and wherein the device transitions from the locked configuration to the unlocked configuration by rotating at least one of said discs.

8. The device as claimed in claim 1, wherein the device is formed of medical grade plastic.
